(19) United States
(12) Patent Application Publication
(10) Pub. No.: US 2024/0024264 A1
(43) Pub. Date: Jan. 25, 2024

(54) FORMULATIONS FOR HEALING OF TRAUMATIC LESIONS OF THE SKIN AND MUCOUS MEMBRANES

(71) Applicants: Rossana Castellana (60%), Trieste (IT); Francesco Piani (40%), Trieste (IT)

(72) Inventors: Rossana Castellana (60%), Trieste (IT); Francesco Piani (40%), Trieste (IT)

(21) Appl. No.: 18/254,884

(22) PCT Filed: Nov. 29, 2021
(86) PCT No.: PCT/EP2021/083345

§ 371 Date: May 30, 2023

Related U.S. Application Data
(60) Provisional application No. 63/119,722, filed on Dec. 1, 2020.

(51) Int. Cl. A61K 31/19; A61K 33/40; A61K 9/06; A61K 47/20; A61K 47/10; A61K 47/44; A61K 47/02; A61K 9/08; A61K 47/18; A61P 17/02

(52) U.S. Cl. CPC A61K 31/19; A61K 33/40; A61K 9/06; A61K 47/20; A61K 47/10; A61K 47/44; A61K 47/02; A61K 9/08; A61K 47/183; A61P 17/02

(57) ABSTRACT

The present invention relates to formulations in the form of a cream, an ointment, a liquid, or a gel and to the use of same to accelerate the healing of wounds and ulcers of mucous membranes and skin. The components of these formulations working together can effectively treat various surgical wounds, spontaneous ulcerations, post traumatic wounds, unhealed wounds, ulcers and bedsores. The formulations of the present invention can accelerate the reparation of wounds and ulcers by itself, reduce inflammation and swelling, relieve pain and itching, and accelerate the epithelialization processes also in cases that are not repairing despite of treatment with antiseptics and fibrinolytics.

### TECHNICAL FIELD

The invention relates to formulations in the form of a cream, an ointment, a liquid, or a gel for use in repairing surgical wounds of skin and mucous membranes. The synergic action of the components of the formulations allows a surprising healing activity of lesions following an odontostomatological intervention like dental extraction, apicectomies, sinus lift, implantology, gingivectomy, periodontal surgery and in the dermatological treatment of slowly healing wounds, spontaneous ulcerations, sores of various origins and necrotic sores including, pyogenic granulomas, chalazion, epulis, fistulas and abscesses.

### BACKGROUND OF THE INVENTION

Disruption of the integrity of skin and mucosal surfaces results in the formation of a wound. Wound healing is a complex biological process which restore the tissue integrity. Physiologically, wound healing can be schematically divided into four distinct phases: haemostasis, inflammation, proliferation and tissue remodelling. Immediately after an injury, the haemostasis process begins: the bleeding is controlled by the aggregation of platelets and the subsequent formation of the fibrin clot stops the bleeding and provides a scaffold for the attachment and proliferation of the cells. After 2-3 days, the inflammatory process progresses into the proliferative phase and fibroblasts are attracted into the wound to synthesize granulation tissue. This granulation tissue allows leukocytes to enter the wound site and once the wound is closed, the immature scar can move on to the final remodelling phase which can last up to a year. The main function of fibroblasts in tissue regeneration is to maintain the physical integrity of connective tissue by producing and remodelling the extracellular matrix. Regardless of the aetiology of the wound, whether acute or chronic, the above described repair processes are similar but while acute wounds, e.g. a surgical incision, usually pass through these phases relatively quickly, wounds undergoing delayed healing up to 12 weeks after the initial insult, often as a result of prolonged pathological inflammation, are defined chronic wounds.

Several insults like biofilm formation, microbial invasion, repeated trauma, ischemia, oedema, venous hypertension and effect of mechanical forces, including pressure, may cause healing impairment. These insults lead to poor migration of keratinocytes and the epithelization is also delayed. In these indolent cases, wound bed preparation remains the standard of therapy.

An effective wound bed preparation may involve enzymatic (J. Ramundo et al., J Wound Ostomy Continence Nurs Actions. May-June 2008; 35(3):273-80; McCarty M S; Adv Wound Care (New Rochelle). 2013 October; 2(8): 438-447) and surgical debridement, resident fibroblast stimulation and stimulation of growth factor release (L. Maddaluno et al., Development (2017) 144, 4047-4060), addition of extraneous growth factors to the wound (M. Yadav et al. Indian Journal of Fundamental and Applied Life Sciences (2012), 2 (2) April-June, 164-172), deployment of bioengineered extracellular matrix (F. Urciolo et al., J. Clin. Med. 2019, 8, 2083), collagen and alginates (B. A. Aderibigbe et al., Pharmaceutics, 2018, 10(2), 42), cultured keratinocyte suspension and even bioengineered dermal preparation (G. Schultz. Wound Repair and Regeneration, April 2003, 11 Suppl 1(s1): S1-28) in any combination and also the stem cell therapy holds promise (N. Kosaric et at., Expert Opinion on Biological Therapy, 19 (6), 2019).

The known treatment options remain only moderately effective and often fail to promote the closure of non-healing wounds in susceptible populations, such as aging and diabetic patients or represent a limited remedy in case of wounds by insect or spider bites (Hindawi Publishing Corporation, Case Reports in Emergency Medicine, Volume 2016, Article ID 7640789). Moreover, some of these approaches, like for example the bioengineered skin substitutes, although representing a valid alternative to autografting, induce skin cells in repairing the wound rather than guiding a regeneration process.

Analogously, the surgical wounding of oral mucosa after surgical incision requires, without complications, long recovery times. In order to restore the original physiology of the oral cavity, up to five weeks are usually required but with some possible complications that could delay the complete healing of the wound. In particular, the normal healing response to tooth extraction results in a significant loss of bone and collapse of the surrounding gingiva. In normal healing, a substantial percentage of extraction sites suffers from postoperative complications.

In fact, after tooth extraction blood clots fill the socket and, after one week, the clots are replaced by granulation tissue; after 3 weeks, the granulation tissue is replaced by collagen, and bone formation begins at the base and the periphery of the extraction socket. At 5 weeks, it is estimated that on average two-thirds of the extraction socket is filled with bone. Epithelium was found to require a minimum of three weeks to completely cover the extraction socket, with some extraction sites requiring up to five weeks to completely cover the socket (Amler M H et al. J Am Dent Assoc. 1960; 61(7), 32-44; Amler M H Oral Surg Oral Med Oral Pathol, 1969, 27 (3), 309-318). Post-operative complications of the above-mentioned normal healing in response to tooth extraction may include dry socket, dysesthesia, severe infection, pain, swelling, trismus and haemorrhage. All these possible complications significantly increase the total healing time from a surgical incision of the oral mucosa.

Trichloroacetic acid alone or in association with other compounds may be used as “chemical peel” (Monheit G D; J. Dermatol. Surg. Oncol. (1989) 15 (9), 945-950). A 50% trichloroacetic (TCA) solution is commonly used as medium-depth chemoexfoliation which is a frequently used to treat fine rhytides, actinic photodamage, hyperpigmentation, and even actinic-related premalignant changes, such as actinic keratoses.

Chloroacetic acid has been disclosed for the treatment of chronic wounds together with an extracellular polymeric substance solvating system surfactant and buffering agent (WO2009152374). Formulations containing trichloroacetic acid and hydrogen peroxide in a pH range of 2.3-2.6 were also proposed for the treatment of pathologies of the skin and mucosa membranes, such as acne, damage caused by the sun and sun freckles (EP1979053) with some limitations due to the fact that hydrogen peroxide, known as mild antiseptic, may be used on the skin to prevent infection of minor cuts, scrapes, and burns but it is endowed with potential negative effects on tissue repairs and should not be used to treat deep wounds, animal, insect or spider bites, or serious burns (Agency for Toxic Substances and Disease Registry, Buford Hwy NE Atlanta-Hydrogen Peroxide).

More recently, the biological effect of hydrogen peroxide during the wound-healing process was reconsidered: in relatively high concentrations (i.e. 3%), hydrogen peroxide displays its strong ability of oxidization and pro-inflammation to disinfect wound tissue (Roy S, et al. Mol Ther 2006; 13:211-220); however, in lower concentrations (i.e. 1%) it shows good antimicrobial effect and skin tolerability (Capizzi R. et al. Br J Dermatol 2004; 151:481-484). On the contrary, an oxidized form of trichloroacetic acid, namely trichloroperoxyacetic acid has never been considered for human use even if in vitro tests confirmed disinfectants properties of diluted aqueous solutions against Bacillus subtilis, Escherichia coli and Staphylococcus aureus (Sita, F. et al. Journal of Hygiene, Epidemiology, Microbiology and Immunology (1968), 12(3), 370-3).

There is thus a need for innovative pharmacological treatments of surgical wounds on skin and mucous membrane, avoiding the above mentioned post-operative complications and allowing an optimal wound bed preparation.

### SUMMARY OF THE INVENTION

In order to solve the above-mentioned problems, the present invention provides formulations for repairing skin and mucous membrane wounds.

The wound healing compositions of the invention comprise:
a first component selected from trichloroacetic or chloroacetic acid in a concentration by weight ranging from 0.08 to 38%;
a second component selected from aqueous hydrogen peroxide in a concentration by weight ranging from 0.01 to 5% or trichloroperoxyacetic acid.

The components of these formulations display a synergic action in the treatment of wounds of skin and mucous membranes.

Trichloroacetic acid and hydrogen peroxide are preferably present in a relative molecular ratio of about 4.34-4.74. Trichloroperoxyacetic acid and trichloroacetic acid are preferably in a relative molecular ratio of about to 3.6-4.0. The formulations of the invention are devoid of the potential negative effects of hydrogen peroxide on tissue Alternatively, the combined use of chloroacetic acid and hydrogen peroxide, instead of trichloroacetic acid and hydrogen peroxide, in the same relative molecular ratio 4.43-4.74, may also be used as components of the above-mentioned formulations.

The formulations of the invention are applied topically in the form of cream, ointment, liquid, gel or similar administration forms. The formulations have a rapid effect, quickly reduce swelling and pain, promote wound healing and reduce the times of recovery of chronic wounds, like chronic ulcers bedsores. The use of the formulations is also effective in treating post-operative complications of normal healing in response to tooth extraction including dry socket, dysesthesia, severe infection, pain, swelling, trismus, haemorrhage and in the treatment of slowly healing wounds, spontaneous ulcerations, sores of various origins and necrotic sores including, pyogenic granulomas, chalazion, epulis, fistulas and abscesses.

### DETAILED DESCRIPTION OF THE INVENTION

The formulations of the invention are characterized by a concentration of hydrogen peroxide aqueous solution (0.01 to 5% of a 5-31%, preferably 29-31%, hydrogen peroxide aqueous solution) remarkably lower than that disclosed in the prior art, particularly in EP 1979053 (50-80% by weight).

The concentration of trichloroacetic preferably ranges from 0.1 to 35% by weight, more preferably from 0.15 to 15% by weight.

Chloroacetic acid and hydrogen peroxide can be alternatively used instead of trichloroacetic acid and hydrogen peroxide, in the same relative molecular ratio of 4.34-4.74 and final concentration 0.08-38% by weight.

The formulations of the invention may be diluted using a 0.9% w/v aqueous NaCl solution. The diluted formulations secure the viability of the cells of the mucosal membrane and of the skin and are useful to accelerate the repair of the surgical wounds, post traumatic wounds, unhealed wounds, ulcers and bedsores. The pH value of the formulations is higher than 2.6, typically in the range from 2.6 to 3.6. Alternatively, trichloroperoxyacetic acid can be prepared in situ by addition of equimolar amounts of hydrogen peroxide to trichloroacetic acid.

The liquid formulations of the invention may contain water in a range from 38 to 99% by weight, EDTA disodium salt in a range from 0.001 to 0.5% by weight, glycerol in a range from 0.025 to 15% by weight, aqueous ammonia (28-32%) in a range from 0.03 to 14% by weight, a thickening and texture agent in a range from 0,005 to 5% by weight and optionally 0.8-0.9% w/v of sodium chloride. Preferred thickening and texture agents include hydroxyethylcellulose, guar gum, locust bean gum, xanthan gum, gelatine and hydroxyethyl acrylate/sodium acryloyldimethyl taurate copolymers. More preferred thickening and texture agents comprise hydroxyethyl acrylate/sodium acryloyldimethyl taurate copolymer (Sepineo™ d.e.r.m). Alternatively, chloroacetic acid and hydrogen peroxide can be used instead of trichloroacetic acid and hydrogen peroxide.

The undiluted ointment formulations of the invention may also contain water in a range between 5 and 12% by weight, sodium laurylsulfate in a range from 0.5 to 1.5% in weight, propylene glycol in a range from 10 to 15% by weight, stearyl alcohol in a range between 20 and 30% by weight, white petrolatum in a range between 20 and 30% by weight, aqueous ammonia (28-32%) in a range between 4 and 6% by weight and optionally one or more preservatives in a concentration between 0.02 and 0.05%. Preferred preservatives include propylparaben, methylparaben, sodium benzoate and ethylhexylglycerin. Optionally, the formulation does not contain any preservative. Chloroacetic acid and hydrogen peroxide or trichloroacetic acid and trichloroperoxyacetic acid can be alternatively used instead of trichloroacetic acid and hydrogen peroxide. These formulations can optionally be administered after dilution with an isotonic 0.9% w/v NaCl aqueous solution.

The undiluted cream formulations of the invention may contain, in addition to the combination of trichloroacetic acid and hydrogen peroxide or of trichloroperoxyacetic acid and trichloroacetic acid in the proportions above defined, water in a range between 20 and 25% by weight, liquid paraffin in a range between 5 and 12% by weight, propylene glycol in a range from 3 to 7% by weight, glycerine in a range from 8 to 12% by weight, aqueous ammonia 28-32% in a range from 10 to 14% by weight and polysorbate 60 in a range from 1 to 2% by weight. Alternatively, chloroacetic acid and hydrogen peroxide of trichloroacetic acid and trichloroperoxyacetic acid can be used instead of trichloroacetic acid and hydrogen peroxide. These formulations can optionally be administered after dilution with an isotonic 0.9% w/v NaCl aqueous solution.

The undiluted gel formulations of the invention may contain ethanol in a range between 25 and 35% by weight, propylene glycol in a range between 11 and 15% by weight, aqueous ammonia (28-32% w/v) in a range of 10-14%, diethylene glycol monoethyl ether in a range from 2 to 5% by weight and myristyl alcohol in a range between 1 and 4%. Alternatively, chloroacetic acid and hydrogen peroxide or trichloroacetic acid and trichloroperoxyacetic acid can be alternatively used instead of trichloroacetic acid and hydrogen peroxide. These formulations can optionally be administered after dilution with an isotonic 0.9% w/v NaCl aqueous solution.

### EXPERIMENTAL SECTION

### Example 1

An ointment for repairing skin and mucous membranes wounds has the following composition:

| Components | % (w/w) |
|---|---|
| Purified water | 11.59 |
| Propylparaben | 0.02 |
| Sodium Laurylsulfate | 1.05 |
| Propylene Glycol | 12.63 |
| Stearyl Alcohol | 26.32 |
| Methylparaben | 0.03 |
| Trichloroacetic acid | 14.60 |
| Hydrogen peroxide 29-31% | 2.21 |
| Aqueous ammonia 28-32% | 5.22 |
| White petrolatum | 26.32 |

This concentrated ointment formulation can be diluted using a 0.9% w/v NaCl aqueous solution to obtain a final liquid formulation with a pH value ≥2.6.

### Example 2

A liquid for repairing wounds of skin and mucous membranes has the following composition (Formulation 14-03-01: 1/400 dilution)

| | Concentrated | 1/10 | 1/100 | 1/200 | 1/400 |
|---|---|---|---|---|---|
| | liquid solution | dilution | dilution | dilution | dilution |
| Components | % (w/w) | % (w/w) | % (w/w) | % (w/w) | % (w/w) |
| Purified water | 38.0 | 93.8 | 99.380 | 99.690 | 99.850 |
| Disodium EDTA | 0.2 | 0.02 | 0.002 | 0.001 | 0.001 |
| Trichloroacetic acid | 33.0 | 3.3 | 0.330 | 0.165 | 0.083 |
| Glycerol | 10.0 | 1.0 | 0.100 | 0.050 | 0.025 |
| Hydrogen peroxide | 5.0 | 0.5 | 0.050 | 0.025 | 0.013 |
| 29-31% | | | | | |
| Aqueous ammonia | 11.8 | 1.2 | 0.118 | 0.059 | 0.030 |
| 28-32% | | | | | |
| Sepineo ™ d.e.r.m. | 2.0 | 0.2 | 0.020 | 0.010 | 0.005 |

The concentrated liquid solution can be diluted up to 1:400 using a 0.9% w/v NaCl aqueous solution to obtain a final liquid formulation with a pH value ≥2.6.

### Example 2a

A liquid for repairing skin and mucous membranes wounds has the following composition:

| Components | % (w/w) |
|---|---|
| Purified water | 41.5 |
| Disodium EDTA | 0.2 |
| Trichloroperoxyacetic acid | 28.3 |
| Trichloroacetic acid | 7 |
| Glycerol | 10 |
| Aqueous ammonia 28-32% | 11.8 |
| Sepineo ™ d.e.r.m. | 2 |

This concentrated liquid solution can be diluted using a 0.9% w/v NaCl aqueous solution to obtain a final liquid formulation with a pH value ≥2.6. Alternatively, trichloroperoxyacetic acid can be prepared in situ by addition of equimolar amounts of hydrogen peroxide to trichloroacetic acid.

### Example 3

A cream for repairing skin and mucous membranes wounds has the following composition

| Components | % (w/w) |
|---|---|
| Purified water | 23.9 |
| Liquid paraffine | 10.0 |
| Trichloroacetic acid | 33.0 |
| Propyleneglycol | 5.0 |
| Hydrogen peroxide 29-31% | 5.0 |
| Aqueous ammonia 28-32% | 11.8 |
| Glycerine | 10.0 |
| Polysorbate 60 | 1.3 |

This concentrated formulation can be diluted using a 0.9% w/v NaCl aqueous solution to obtain a final liquid formulation with a pH value ≥2.6.

### Example 4

A gel for repairing skin and mucous membranes wounds has the following composition:

| Components | % (w/w) |
|---|---|
| Ethanol | 30.8 |
| Trichloroacetic acid | 33.0 |
| Propylene glycol | 13.9 |
| Hydrogen peroxide 29-31% | 5.0 |
| Aqueous ammonia 28-32% | 11.8 |
| Diethylene glycol monoethyl ether | 3.5 |
| Myristyl alcohol | 2.1 |

This concentrated formulation can be diluted using a 0.9% w/v NaCl aqueous solution to obtain a final liquid formulation with a pH value ≥2.6.

### Comparative Example 1

The only difference from Example 2 (Formulation 14-03-01) is that the components do not contain hydrogen peroxide.

Formulation 14-04-03

| Components | % (w/w) |
|---|---|
| Trichloroacetic acid | 33.0 |
| Glycerol | 10.0 |
| Purified water | 43.0 |
| Aqueous ammonia 28-32% | 11.8 |
| Sepineo ™ d.e.r.m. | 2.0 |
| Disodium EDTA | 0.2 |

This concentrated formulation was diluted 1:400, using a 0.9% w/v NaCl solution to obtain a final liquid formulation with a pH value ≥2.6.

### Comparative Example 2

Formulation 14-04-05

| Components | % (w/w) |
|---|---|
| Glycerol | 10.0 |
| Hydrogen peroxide 29-31% | 5.0 |
| Purified water | 84.6 |
| Aqueous ammonia 28-32% | 0 |
| Sepineo TM d.e.r.m. | 0.2 |
| Disodium EDTA | 0.2 |

This concentrated liquid solution was diluted 1:400, using a 0.9% w/v aqueous NaCl solution to obtain a final liquid formulation with a pH value ≥2.6.

The liquid formulations 14-03-01 (TCA plus H₂O₂), 14-04-03 (TCA only) and 14-04-05 (H₂O₂ only) were then biologically evaluated in in vivo and in vitro tests. These tests confirmed the synergic action of trichloroacetic acid and hydrogen peroxide in accelerating the healing of wounds and ulcers of mucous membranes and skin, even after dilution up to 400 time with a 0.9% NaCl aqueous solution and at a pH value ≥2.6.

Experimental In Vitro and In Vivo Tests

Experimental Protocol

This study was divided in two steps: in the first, the effectiveness of solutions was verified on an in vitro cell model using NHEK cells (normal human epidermal cell); in the second the effectiveness of solutions was carried out on a mouse animal model mimicking a slow-healing human wound. The solutions tested in both steps included the following combinations:
14-03-01 (TCA plus H₂O₂)
14-04-03 (TCA only)
14-04-05 (H₂O₂ only)
and their effects were compared to an untreated control sample.

In an in vitro model, NHEK cells were used to test the efficacy of the solutions in a dose-response study to exclude any cytotoxic effects analyzing cell viability by MTT (3-(4,5-dimethylthiazol-2-yl)-2,5-diphenyl tetrazolium bromide) test. Since the combinations of substances have never been described in the literature, the range tested was based on TCA concentration reported in literature [Yang H, et al., Toxicol In Vitro. 2011 December; 25(8):1638-43]. In particular, 1:100 (20 mM); 1:200 (10 mM); 1:400 (5 mM) and 1:800 (2.5 mM) diluted with sterile physiological solution (0.9% NaCl) were tested. 3 different stimulation protocols were selected, as described below:
1 treatment only that is left up to max 6 days (total 1 stimulus), protocol A
1 treatment/day that is carried out max 6 days (total of 5 stimuli), protocol B
1 treatment/alternate days carried out max 6 days (total 3 stimuli), protocol C.

Since the data highlighted the minimum effective dose and the importance of administering an adequate dose, in the subsequent experiments only the 5 mM concentration was tested using only the protocols A and C. Subsequently, tests were carried out to evaluate cell proliferation (Crystal Violet assay), migration (wound assay) and production of reactive oxygen species (ROS).

Migration and re-epithelization were investigated in an in vivo model using male C57BL/6JOlaHsd mice, excluding any toxic effects induced by the substances tested in protocol A and C. Before administration, the mice were depilated on the right side of the back spine. The depilated area was about 2×2 cm. The bedsore model was squeezed in the depilated area by intermittent mechanical extrusion to simulate the natural formation process of bedsores.

In particular,
14-03-01 (TCA plus H₂O₂)
14-04-03 (TCA only)
14-04-05 (H₂O₂ only)
were used at 1:400 dilutions prepared in sterile physiological solution (0.9% NaCl) to treat skin wounds. At the end of each treatment, a planimetric analysis of the wounds was performed and wound areas were collected to make histological analysis as E/E (hematoxylin/eosin) and Masson staining.

Methods: In Vitro Model

Cell Culture

Normal human epidermal keratinocyte cells (NHEK) purchased from Lonza (Basel, Switzerland) were cultured in EpiLife medium (Gibco, Thermo Fisher Scientific, Waltham, MA, USA) containing 60 μM Ca2+ and HKGS (Gibco, Thermo Fisher Scientific) in a 5% CO₂ incubator (Thermo Fisher Scientific) at 37° C. The medium was replaced every two days, and the cells were used at 70% to 80% confluence [Jia, T et al., Molecules 2019, 24, 3156]. Experiments were conducted at passages 3-6.

Cell Viability

After each stimulation, the NHEK cells were washed with sterile PBS 1× and incubated with DMEM (Dulbecco s modified eagle medium) without red phenol and FBS (fetal bovine serum) containing 1% MTT dye (MTT-Based In Vitro Toxicology Assay Kit; Sigma-Aldrich) for 2 h at 37° C. and 5% CO₂ [Uberti, F et al., J Cardiovasc Pharmacol 57: 246-258.]. Cell viability was determined by measuring absorbance using a spectrometer (VICTORX4 multi-label plate reader) at 570 nm with correction at 690 nm and calculated by comparing the results to control cells (100% viable).

Crystal Violet

After each treatment the cells were fixed with 1% glutaraldehyde (Sigma-Aldrich) for 15 min at room temperature, washed, and stained with 100 μL 0.1% aqueous crystal violet (Sigma-Aldrich) for 20 min at room temperature. 100 μL microliters of 10% acetic acid were added to multi-well plates and mixed before reading the absorbance at 595 nm using a spectrometer (VICTORX4 multi-label plate reader). The estimated number of cells was calculated by comparing the results to the control cells (control TO), examined on the first treatment, and the variation of the untreated cells was also reported [Uberti F, Cells Tissues Organs. 2017; 203(4):215-230. Epub 2016 Nov. 25.].

Wound Assay In Vitro

A scratch wound healing assay was performed as previously described [Uberti F, et al., Cells Tissues Organs. 2017; 203(4):215-230. Epub 2016 Nov. 25.] in confluent monolayer cells using a sterile p200 pipette tip. Afterwards, the cells were stimulated with different preparations in different protocols (A and C) and monitored for 6 days. After each time point, repopulation of the wounded areas was observed under a phase contrast microscope (Leitz, Germany). Using the ImageJ image-processing program, the size of the denuded area was determined at each time point from digital images taken at 6 different areas. The results are expressed as means±SD (%) of migrated cells.

ROS Production

The rate of superoxide anion release was measured using a standard protocol based on reduction of cytochrome C. In both treated and untreated cells, 100 μL cytochrome C were added and in another sample 100 μL superoxide dismutase were also added for 30 min in an incubator (all substances were from Sigma-Aldrich). The absorbance in culture supernatants was measured at 550 nm using a spectrometer (VICTORX4 multilabel plate reader) and O2 was expressed as the mean±SD (%) of nanomoles per reduced cytochrome C per microgram of protein compared to the control [Uberti F, et al., Cells Tissues Organs. 2017; 203(4):215-230. Epub 2016 Nov. 25].

Results of the In Vitro Tests

Dose-Response Study of Cell Viability on NHEK Cells.

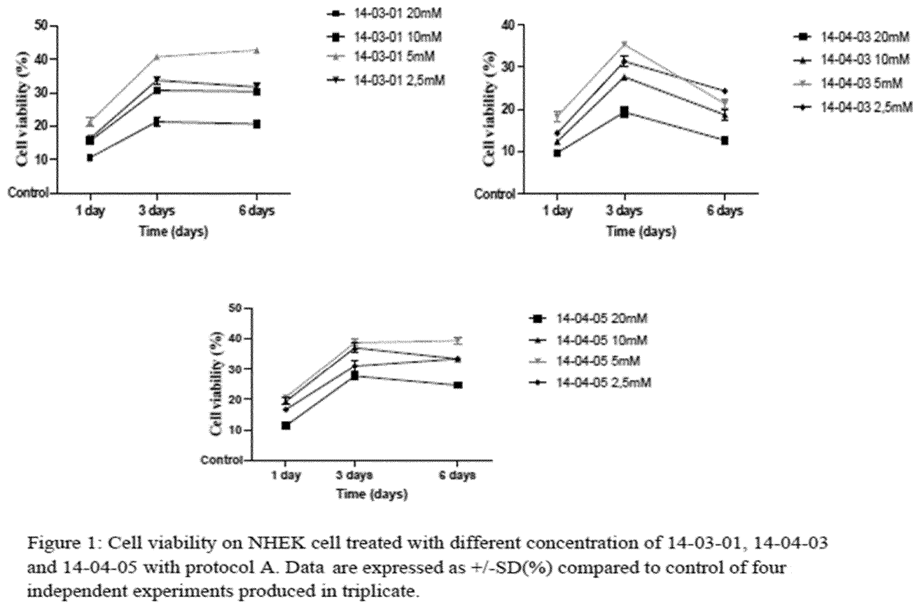

The dose-response study was conducted evaluating the best concentration within the range found in the literature. This test allowed to determine the metabolism of the mitochondrion by analyzing the administration protocols. As shown in FIG. 1 the protocol A showed a dose-response effect for all substances with a peak around three days of stimulation; this effect can stabilize only with 14-03-01 and the main effect was observed with 5 mM (1:400 dilution).

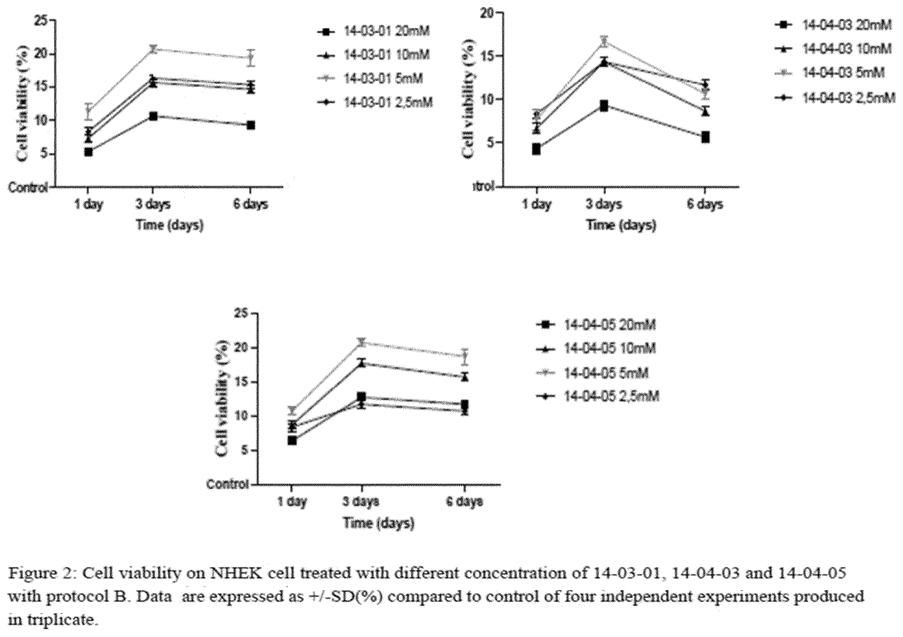

The same tests were also conducted with protocol B (FIG. 2) in which the tested composition was administered one treatment/day for 6 days. Also, in this case the best results were obtained by 5 mM (p<0.05) for all tested solutions. Considering that the results are “lower” than those observed in protocol A, it is assumed that the daily stimulation is excessive but not toxic. The plateau phase tends to be slightly descending, demonstrating that protocol B is excessive and for this reason protocol B was replaced by protocol C (1 treatment/alternate days carried out max 6 days) in subsequent experiments.

Proliferation Analysis

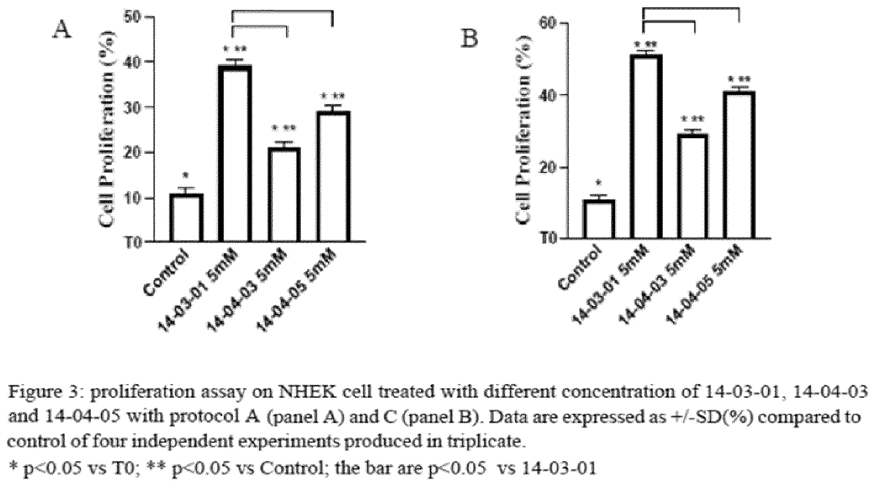

The proliferation assay was conducted using crystal violet on NHEK cells treated with the substances according to protocols A and C. As shown in FIG. 3, 14-03-01 significantly increased cell proliferation compared to other compositions (p<0.05), particularly one more time compared to 14-04-03 and about 33% to 14-04-05. In addition, these effects are evident in both protocols, but the main effect was obtained by protocol C (p<0.05 vs A). This is important because by increasing the number of cells it is possible that they can also migrate; in addition, these data suggested an activity of 14-03-01 higher than that of the individual components (p<0.05).

Effects of 14-03-01, 14-04-03 and 14-04-05 on NHEK Migration

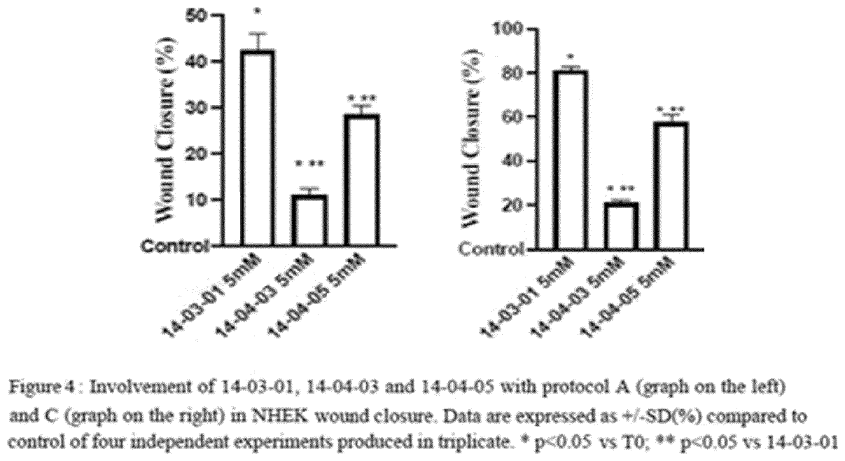

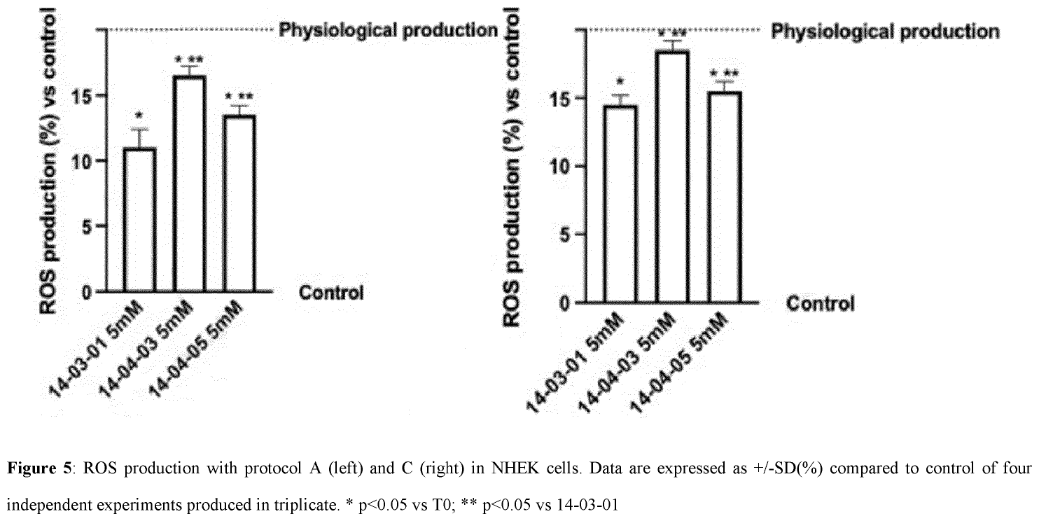

The effect of the different formulations on NHEK migration in wound healing were evaluated (FIG. 4). Our results demonstrated an improvement in migration activity (p<0.05) in NHEK cells treated with 14-03-01 (both protocol A and C) compared to control and compared to 14-04-03 (about 3 times more) and 14-04-05 (about 60%). This is a validated method to characterize various factors involved in migration. The data confirm what observed during proliferation analysis.

Effects of Formulations on ROS Production

Since conflicting data are reported in literature on the ROS role on healing, further experiments were carried out to explore the role of ROS in this context. As shown in Figure despite the presence of H₂O₂, ROS production does not increase above the physiological share of the tested substances; this is certainly important to ensure the effectiveness observed in previous tests. Obviously, the compound with hydrogen peroxide has a higher ROS production than the other substances in both protocols but 14-03-01 is able to remain below the physiological level of ROS compared to the control and compared to 14-04-03 (about 35%) and compared to 04-14-05 (about 23%), confirming once again the synergistic effect of TCA and hydrogen peroxide to neutralize the negative consequence of the two components administered individually. ROS production is dose dependent since protocol C gives higher results than protocol A but still physiological.

Methods and Results of the In Vivo Tests

In order to validate the data obtained in vitro, in vivo tests were performed using 60 animals (C57BL/6JOlaHsd mouse) of 8 weeks weighing 20-25 g. The method allows to compare different preparations with a specific control by monitoring the wound healing area.

Evaluation of Wound Healing Area

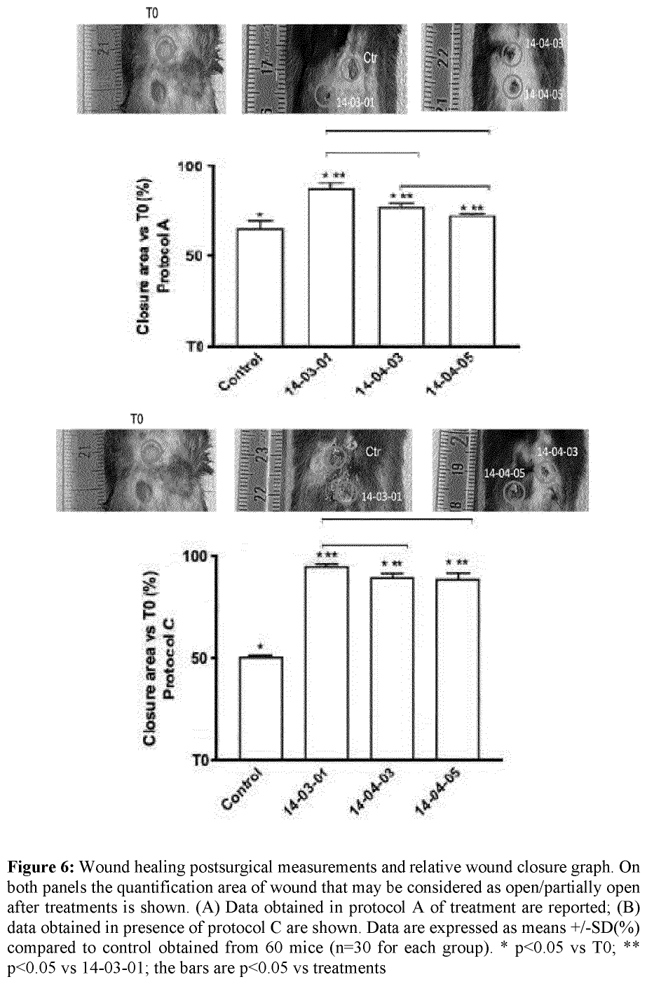

The experimental model of wound healing was performed in male mice treated for 8 days using 14-03-01, 14-04-03 and 14-04-05. As reported in FIG. 6, in protocol A, 14-03-01 induced a greater closure area than control (p<0.05) and then compared to 14-04-03 and 14-04-05 (14% and 33% respectively). This effect was also statistically significant (p<0.05) in comparison with untreated wounds. In protocol C the presence of 14-03-01 induced a faster closure in comparison to control (about 98%), 14-04-03 (about 2%) and 14-04-05 (about 4%).

These findings have been confirmed by morphological analysis with E/E and Masson analysis in which the formulation 14-03-01 seems to be a better choice. In all treatments of protocol A, the granulation tissue seemed to be still very active, and the healing was at an early stage. In particular, 14-03-01 showed a quite evident capacity to repair wound and re-epithelization was almost complete. In contrast, protocol C showed that the mice treated with 14-03-01 presented a complete epithelialization of the wound compared to control, to 14-04-03 and to 14-04-05, respectively. Epithelialization appeared without any ulcer, with the presence of inactive granulation tissue, with collagen remodeling and angiogenesis; for this reason, it is possible to envisage a restitutio ad integrum.

1. Wound healing compositions in form of cream, ointment, liquid or gel comprising:
a first component selected from trichloroacetic or chloroacetic acid in a concentration by weight ranging from 0.08 to 38%;
a second component selected from aqueous hydrogen peroxide in a concentration by weight ranging from 0.01 to 5% or trichloroperoxyacetic acid.

2. Compositions according to claim 1 comprising trichloroacetic acid and hydrogen peroxide.

3. Compositions according to claim 1 comprising trichloroacetic acid and trichloroperoxyacetic acid.

4. Compositions according to claim 1 comprising chloroacetic acid and hydrogen peroxide.

5. Compositions according to claim 2 wherein the relative molar ratio between trichloroacetic acid and hydrogen peroxide is from 4.34 to 4.74.

6. Compositions according to claim 3 wherein the relative molecular ratio between trichloroperoxyacetic acid and trichloroacetic acid is from 3.6 to 4.0.

7. Compositions according to claim 4 wherein the relative molar ratio between chloroacetic acid and hydrogen peroxide is from 4.34 to 4.74.

8. Compositions according to claim 1, wherein the hydrogen peroxide aqueous solution is a 5-31% aqueous solution.

9. Compositions according to claim 1, having a pH value from 2.6 to 3.6.

10. Compositions according to claim 1, in form of a cream containing 20-25% w/w water, 5-12% w/w liquid paraffin, 3-7% w/w propylene glycol, 8-12% w/w glycerin, 10-14% w/w of a 28-32% w/w aqueous ammonia solution and 1-2% w/w polysorbate 60.

11. Compositions according to claim 1, in form of an ointment containing 5-12% w/w water, 0.5-1.5% w/w sodium laurylsulfate, 10-15% w/w propylene glycol, 20-30 w/w stearyl alcohol, 20-30% w/w white petrolatum, 4-6% w/w of a 28-32% w/w/ aqueous ammonia solution and optionally one or more preservatives in a concentration between 0.02 and 0.05%.

12. Compositions according to claim 11 wherein no preservatives are used.

13. Compositions according to claim 1, in form of a liquid containing 38-99% w/w water, 0.001-0.5% w/w EDTA disodium salt, 0.025-15% w/w glycerol, 0.03-14% w/w of a 28-32% aqueous ammonia solution and, 0.005-5% of a thickening and texture agent.

14. Compositions according to claim 1, claims 1-9 in form of a gel containing 25-35% w/w ethanol, 11-15% w/w propylene glycol, 10-14 w/w of a 28-32% an aqueous ammonia solution, 2-5% w/w diethylene glycol monoethyl ether and 1-4% myristyl alcohol.

15. Compositions according to claim 1, diluted with a 0.9% NaCl aqueous solution.

16. Compositions according to claim 1, wherein trichloroperoxyacetic acid is prepared in situ by addition of hydrogen peroxide to trichloroacetic acid.

17. method of treating unhealed wounds, ulcers and bedsores, of post-operative complications to tooth extraction including dry socket, dysesthesia, severe infection, pain, swelling, trismus and haemorrhage, of slowly healing wounds, spontaneous ulcerations, sores of various origins and necrotic sores including, pyogenic granulomas, chalazion, epulis, fistulas and abscesses in patients in need thereof with the composition of claim 1, said method comprising
treating said patients with a pharmaceutical effective amount of said composition.

18. Compositions according to claim 1, wherein the hydrogen peroxide aqueous solution is a 29-31% aqueous solution.
